(19) United States
(12) Patent Application Publication
(10) Pub. No.: US 2010/0036361 A1
(43) Pub. Date: Feb. 11, 2010

(54) SYSTEM AND METHOD FOR DELIVERING MULTIPLE IMPLANTS INTO LUNG PASSAGEWAYS

(75) Inventors: Hoang Nguyen, San Jose, CA (US); Niyazi Beyhan, Santa Clara, CA (US); Son Gia, San Jose, CA (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: PULMONx, Palo Alto, CA (US)

(21) Appl. No.: 12/474,179

(22) Filed: May 28, 2009

Related U.S. Application Data
(60) Provisional application No. 61/074,261, filed on Jun. 20, 2008.

(51) Int. Cl. A61M 31/00; A61M 5/315

(52) U.S. Cl. 604/500; 604/218

(57) ABSTRACT

A system for delivering multiple implants into lung passageways is disclosed. The system comprises a catheter configured to receive and store a plurality of expandable implants, and an actuatable delivery tool coupled to the proximal end of the catheter. The system expels an implant of the plurality of implants with each actuation of the delivery tool. The delivery tool comprises a plunger element which extends through the catheter from the proximal to the distal end and allows the delivery tool to mechanically communicate with the implants. In one embodiment, the system comprises a spring-loaded slidable element, and a user compresses the slidable element to actuate the delivery tool. In another embodiment, the system comprises a rotation rod, and the user moves the rotation rod in a distal direction to actuate the delivery tool.

### CROSS-REFERENCE TO RELATED APPLICATION

This application claims the benefit of Provisional Application No. 61/074,261 (Attorney Docket No. 017534-004900US), filed on Jun. 20, 2008, the full disclosure of which is incorporated herein by reference.

### BACKGROUND OF THE INVENTION

1. Field of the Invention

The present invention relates generally to the delivery of implants into the body, and more specifically to a system and method for delivering multiple pulmonary implants into lung passageways.

2. Description of the Background Art

Chronic obstructive pulmonary disease is a significant medical problem affecting 16 million people or about 6% of the U.S. population. Specific diseases in this group include chronic bronchitis, asthmatic bronchitis, and emphysema. While a number of therapeutic interventions are used and have been proposed, none are completely effective, and chronic obstructive pulmonary disease remains the fourth most common cause of death in the United States. Thus, improved and alternative treatments and therapies would be of significant benefit.

Lung function in patients suffering from some forms of chronic obstructive pulmonary disease can be improved by reducing the effective lung volume, typically by resecting diseased portions of the lung. Resection of diseased portions of the lungs both promotes expansion of the non-diseased regions of the lung and decreases the portion of inhaled air which goes into the lungs but is unable to transfer oxygen to the blood. Lung reduction is conventionally performed in open chest or thoracoscopic procedures where the lung is resected, typically using stapling devices having integral cutting blades. Although these procedures appear to show improved patient outcomes and increased quality of life, the procedure has several major complications, namely air leaks, respiratory failure, pneumonia and death. Patients typically spend approximately 5-7 days in post-op recovery with the majority of this length of stay attributed to managing air leaks created by the mechanical resection of the lung tissue.

In an effort to reduce such risks and associated costs, minimally or non-invasive procedures have been developed. Endobronchial Lung Volume Reduction (ELVR) allows the physician to use a catheter-based system to reduce lung volumes. ELVR can be achieved by placement of a pulmonary implant or lung implant within a lung segment. The pulmonary implant, such as that described in US Patent Application Publication No. 20060135947, restricts air flow in the inhalation direction while permitting air flow in the exhalation direction. The pulmonary implants thus allow air to be expelled from the diseased region of the lung while blocking re-inflation of the diseased region. This causes the lung segment to collapse over time, reducing the total lung volume. By creating areas of selective atelectasis or reducing the total lung volume, the physician can enhance the patient's breathing mechanics by creating more space inside the chest wall cavity for the more healthy segments to breathe more efficiently. Thus far, this method involves positioning one implant at a time within the pulmonary passageways. The single implant is loaded into a catheter, which is in turn introduced into the trachea through a bronchoscope. If multiple implants are necessary, the catheter is withdrawn, a second implant is loaded onto the same or a different catheter, and the catheter is reintroduced into the bronchoscope to deliver the second implant.

While some methods and devices for delivering multiple stents exist in the coronary field, such as disclosed in U.S. patent application Ser. No. 10/412,714, they are not suitable for use within the pulmonary space. For example, the handle requires the user to carefully manipulate one component (e.g. the catheter sheath) with one hand while stabilizing another component (e.g. the catheter rod) with the other hand. This would be unsuitable for pulmonary ELVR procedures that additionally require a user to manipulate and navigate a bronchoscope. Additional improvements are desired. In particular, a delivery system is desired which can position multiple implants within one or more desired segments of a body passageway with high accuracy, without requiring that the delivery catheter be withdrawn, reloaded and reinserted for each implant delivery. Such a delivery system should provide a way to expel multiple implants out of a delivery catheter, one implant at a time, by repeating a simple mechanical movement of the delivery system even with one hand and without requiring that the user carefully maneuver a guidewire or such other element. Such a delivery system should be easy to use, allow interchangeability of a variety of instruments, and use conventional bronchoscopes to deliver the implants to the passageways. Such utilization should be easy to operate and not interfere with additional therapies which utilize the bronchoscope. Additionally, such a delivery system could also be used for delivering multiple non-pulmonary implants to any bodily lumen. At least some of these objectives are met by the current invention.

### BRIEF SUMMARY OF THE INVENTION

The present invention discloses a system for delivering multiple implants into lung passageways. The system comprises a catheter having a proximal and distal end, the distal end configured to receive and store a plurality of expandable implants, and an actuatable delivery tool coupled to the proximal end of the catheter. The system is configured to expel an implant of the plurality of implants out of the distal end of the catheter with each actuation of the delivery tool. The system comprises a plunger element coupled to the delivery tool which extends through the catheter, allowing the delivery tool to mechanically communicate with the plurality of implants and expel them with each actuation of the delivery tool.

In one embodiment, the system comprises a slidable element which can be compressed by a user to actuate the delivery tool. The slidable element comprises a spring-activated mechanism having one or more springs for incrementally moving the plunger element forward, with each incremental forward movement of the plunger element expelling an implant. The plunger element comprises indentations which engage with the springs to allow forward incremental movements of the plunger element.

In another embodiment, the delivery tool comprises a rotation rod for actuating the delivery tool. Moving the rotation rod in a distal direction causes incremental forward movement of the plunger element. An implant is expelled with each incremental forward movement of the plunger element. The rotatable rod is configured to move slidably within a housing. The housing comprises a non-linear groove, and the rotation rod comprises a pin configured to fit within and move slidably and incrementally along the groove. The groove comprises major sub-grooves arranged coaxially with the rotation rod and minor sub-grooves arranged non-coaxially with the rotation rod, with the lengths of the major sub-grooves corresponding to the lengths of the implants, and the minor sub-grooves serving as stops between successive implant deliveries.

### BRIEF DESCRIPTION OF THE DRAWINGS

The invention has other advantages and features which will be more readily apparent from the following detailed description of the invention and the appended claims, when taken in conjunction with the accompanying drawings, in which:

FIG. 1a shows an exemplary delivery system comprising a delivery tool and a delivery catheter.

FIG. 1b shows the exemplary delivery tool of FIG. 1a in more detail.

FIG. 1c shows the distal end of the delivery catheter, with multiple implants loaded into the catheter lumen.

FIGS. 2a through 2g illustrate the detail and usage of one of the embodiments of the present invention.

FIGS. 3a through 3g illustrate the detail and usage of a second embodiment of the present invention.

### DETAILED DESCRIPTION OF THE INVENTION

Although the detailed description contains many specifics, these should not be construed as limiting the scope of the invention but merely as illustrating different examples and aspects of the invention. It should be appreciated that the scope of the invention includes other embodiments not discussed in detail. Various other modifications, changes and variations which will be apparent to those skilled in the art may be made in the arrangement, operation and details of the method and apparatus of the present invention disclosed herein without departing from the spirit and scope of the invention as described here.

The present invention contemplates delivery of multiple implants into the lung using a delivery catheter, without the need to retract, reload and reinsert the catheter for each implant delivery. This is achieved by using a delivery system capable of carrying multiple implants to a lung compartment and deploying the implants, one implant at a time, at one or more locations in the lung compartment. Such compartments could be an entire lobe, a segment, or a sub-segment and beyond (hereinafter referred to as compartments).

Advantageously, the ability to carry multiple implants to a site and deliver them one by one considerably reduces the total time needed to deliver the multiple implants, as it eliminates the need to go through a cycle of retracting, loading, and reinserting a delivery catheter for the delivery of each individual implant.

As should be obvious to one of ordinary skill in the art, the present embodiments can be used with any implants that are delivered bronchoscopically for inducing atelectasis, as well as implants that can be delivered using virtual bronchoscopy techniques. Such implants may be restrictive or occlusive in nature, or valve-based. Such implants may comprise an air flow channel and may be configured to restrict air flow in the inhalation direction, while allowing air flow in the exhalation direction.

Additionally, the present systems and methods for delivering multiple implants may be used to place multiple implants in other body lumens, such as in the peripheral vasculature, the cerebral vasculature, and in other ducts, such as the biliary duct, the fallopian tubes, and the like. The term implant is thus defined to also include any of the wide variety of expandable prostheses and scaffolds which are designed to be intraluminally introduced to a treatment site and expanded in situ to apply a radially outward force against the inner wall of the body lumen at that site.

The delivery system of the present embodiments comprises a delivery tool configured to receive a delivery catheter. Alternatively, the delivery system comprises both the delivery tool and a delivery catheter. The delivery catheter (hereinafter also referred to as a catheter) has a distal end which is configured to receive and store multiple implants for subsequent delivery. The implants are loaded into the lumen of the delivery catheter, for example by sequential repetition of the implant loading method disclosed in PCT Patent Application Serial No. PCT/US2008/056289, or by any other method.

The proximal end of the delivery catheter is permanently or detachably coupled to a delivery tool. The delivery tool comprises a plunger element that extends through the lumen of the catheter from the proximal end to the distal end of the catheter, thereby allowing the delivery tool to be in mechanical communication with the loaded implants at the distal end of the catheter. Alternatively, the delivery tool comprises a plunger element that mechanically communicates with a component within the inner lumen of the catheter, said component being in mechanical communication with the loaded implants at the distal end of the catheter.

The delivery tool is configured to be actuatable by a user (using compression or rotation, as described below), with each actuation of the delivery tool causing an incremental forward movement of the plunger element (i.e., movement in the distal direction). The forward movement of the plunger element through the catheter lumen in turn forces the deployment of the most distally located implant within the catheter, expelling the implant out of the distal end of the catheter and into the deployment site (e.g., a lung passageway). The implants are made of a shape-memory material and are compressed when stored, and they expand to their intended shape when expelled. By repeatedly actuating the delivery tool and causing incremental forward movements of the plunger element, a user can introduce one implant at a time from the delivery catheter into the deployment site.

There are several ways to actuate the delivery tool. In one embodiment, the delivery tool comprises a sliding component that can be compressed by a user, with a spring-loaded mechanism causing the sliding component to return to its original position and simultaneously moving the plunger element incrementally forward through the delivery catheter lumen. In another embodiment, the delivery tool comprises a rotational mechanism that can be rotated by a user, with the rotational mechanism in turn incrementally moving the plunger element forward through the delivery catheter lumen. In each embodiment, the delivery tool is configured such that distance of each forward movement of the plunger element is sufficient to deploy one implant. Thus, the distal end of the catheter is positioned at a delivery location, and the delivery tool is actuated to deploy one implant. The catheter can then be positioned at a second deployment location, and the delivery tool can be actuated once again to deploy a second implant. The process can thus be repeated such that multiple implants are introduced at one or more deployment locations.

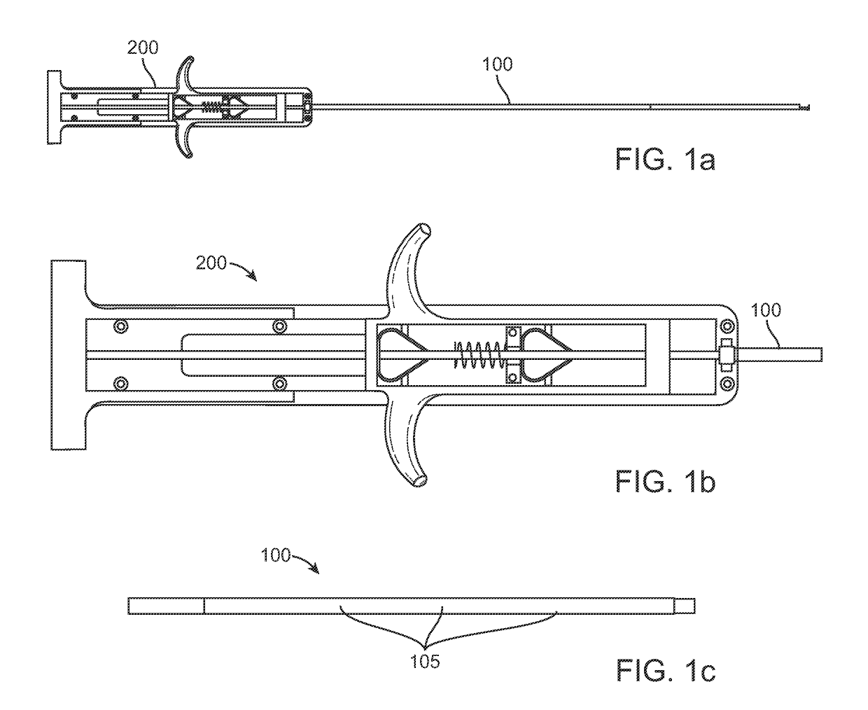

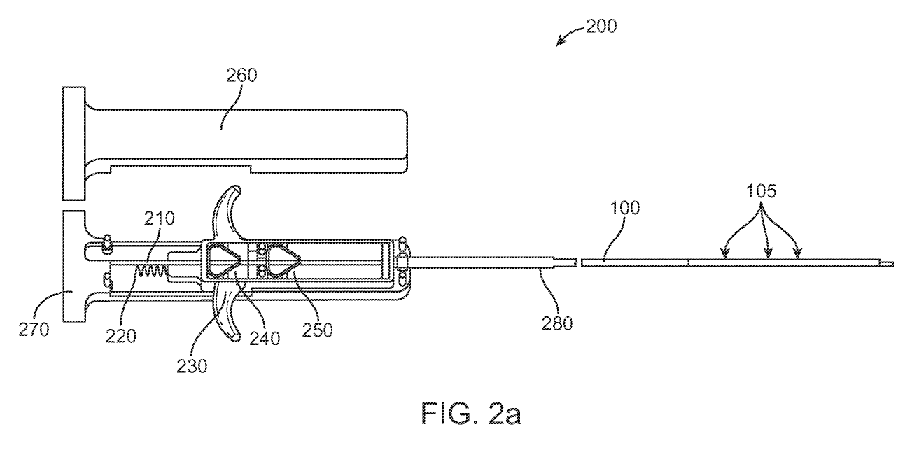

FIG. 1a shows an exemplary delivery system comprising a delivery catheter 100 and a delivery tool 200. The proximal end of the catheter 100 is permanently or detachably attached to the distal end of the delivery tool 200. FIG. 1b shows a close-up of the delivery tool 200, and FIG. 1c shows the delivery catheter 100 storing a plurality of implants in a compressed state.

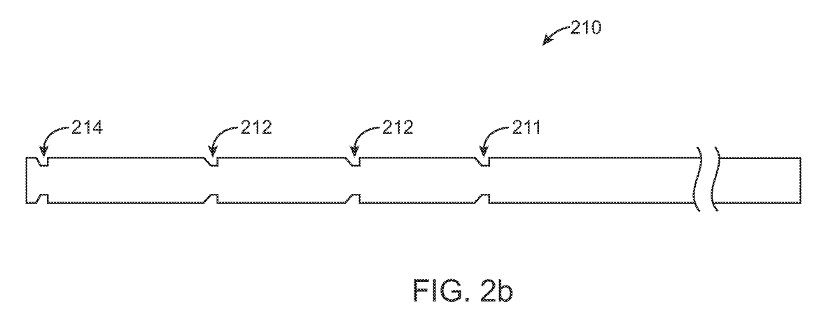

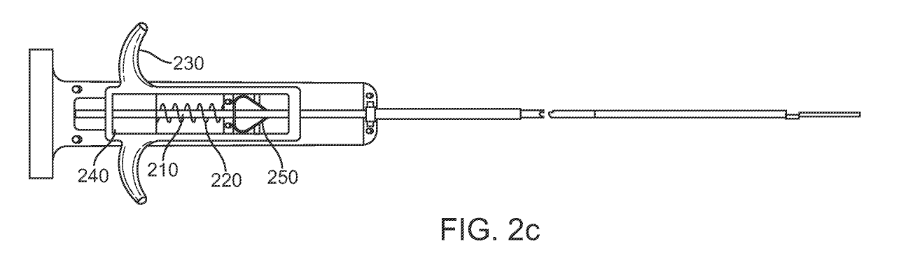

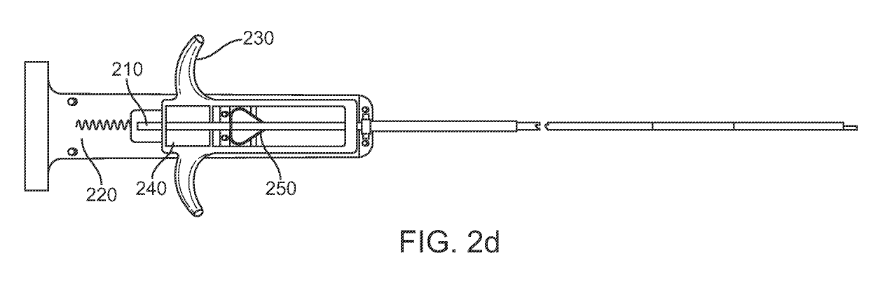

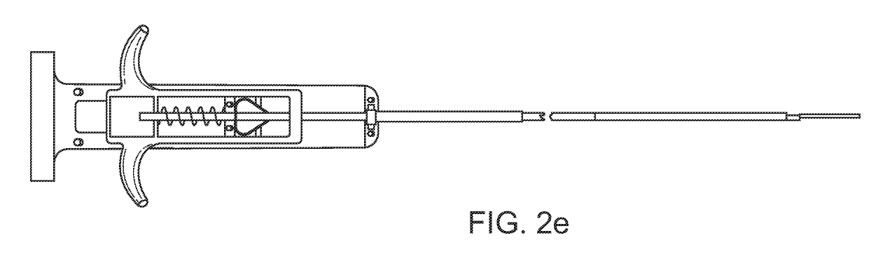

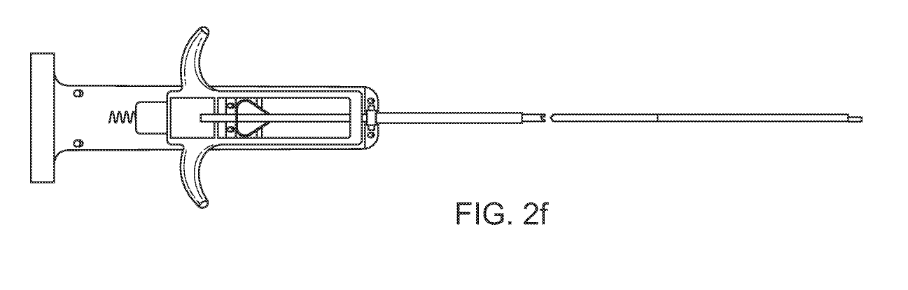

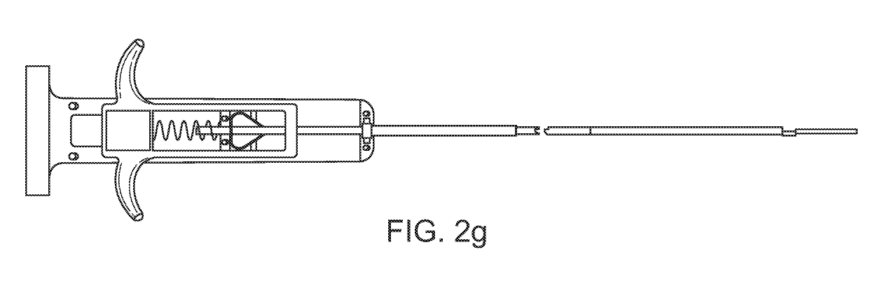

FIGS. 2a through 2h show the operational sequence of one embodiment of the delivery tool 200, comprising a plunger element that moves forward via a spring-loaded mechanism. As shown in FIG. 2a, the delivery tool 200 comprises a plunger element 210, a coil spring 220, a slidable element 230 optionally comprising finger rests, a moving leaf spring 240, and a stationary leaf spring 240. The coil spring 220 is suspended between the moving leaf spring 240 and the stationary leaf spring 250 and is co-axial with the plunger element 210. The components are contained within a back cover 260 and a front cover 270. As described above, the delivery tool 200 is configured to receive and couple to the proximal end of catheter 100. FIG. 2a also shows the distal end of catheter 100 preloaded with one or more implants 105. An optional catheter strain relief element 280 protects the proximal end of the catheter 100 from strains and kinks at the site of coupling with the delivery tool 200.

FIG. 2b shows an embodiment of the plunger element 210 in more detail. The plunger element 210 comprises a plurality of indentations. In the example embodiment shown in FIG. 2b, four indentations are shown on the plunger element 210, which are labeled 211 through 214. The indentations are configured to receive a leaf spring 240 or 250. Additionally, the indentations are configured such that forward movement of the moving leaf spring 240 will engage with an indentation and move the plunger element 210 forward. The stationary leaf spring 250, when engaged with an indentation, prevents the backward movement of the plunger element 210 without impeding its forward movement. This allows the moving leaf spring 240 to subsequently move backward and return to its original position, without causing the plunger element 210 to move backward with it.

FIG. 2a shows the starting position of the delivery tool 200. At the starting position, the stationary leaf spring 250 is engaged within a first indentation (such as the indentation labeled 211 in FIG. 2b) and the moving leaf spring 240 is engaged within a neighboring and more proximally located indentation (such as the indentation labeled 212 in FIG. 2b).

To deploy the first implant, a user compresses the slidable element 230, as shown in FIG. 2c (user's hand not shown). As the slidable element 230 is compressed, the coil spring 220 extends, and the moving leaf spring 240 is drawn back in the proximal direction along plunger element 210 to a more proximally located indentation (such as from indentation 212 to indentation 213, as shown in FIG. 2b).

As the user releases the slidable element 230, the coil spring 220 contracts, pulling the slidable element 230 in the distal direction and to the front position, as shown in FIG. 2d. Simultaneously, the moving leaf spring 240, which is still caught within indentation 213, moves forward and pushes the plunger element 210 along with it in the distal direction. The motion of the plunger element 210 expels and deploys the most distally located implant 105 at the distal end of the catheter 100. At the end of the movement of the slidable element 230 and as shown in FIG. 2d, the stationary leaf spring 250 catches at the indentation 212, at which point the delivery tool 200 has returned to its initial position. As will be obvious to one of ordinary skill in the art, the delivery tool 200 may be configured such that the coil spring 220 is compressed when the user compresses the slidable element 230 and expands when the user releases the slidable element 230.

At this point and as shown in FIG. 2e, the slidable element 230 may be compressed again to deploy the second implant. The moving leaf spring 240 moves back along the plunger element 210 to the next proximally located indentation 214, and the cycle starts over again, as shown in FIGS. 2f and 2g. This process may be repeated as many times as desired to deploy subsequent implants, and is only limited by the number of implants loaded and stored in the catheter 100 and by the length of and number of indentation in the proximal portion of the plunger element 210.

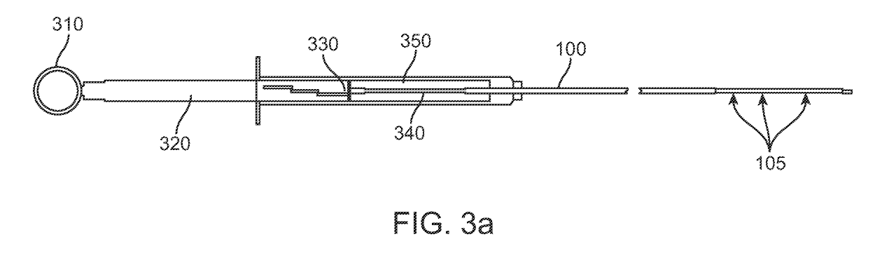

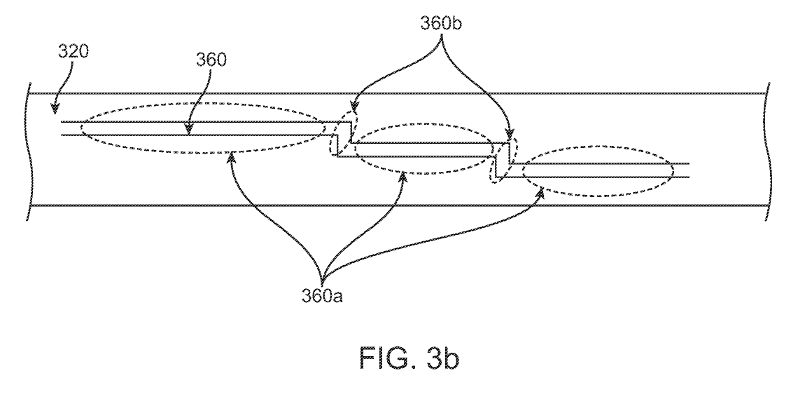

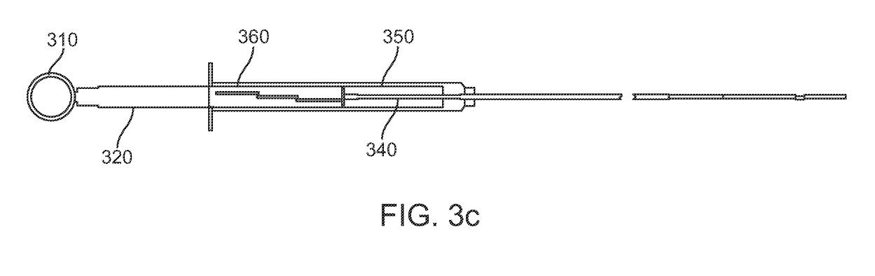

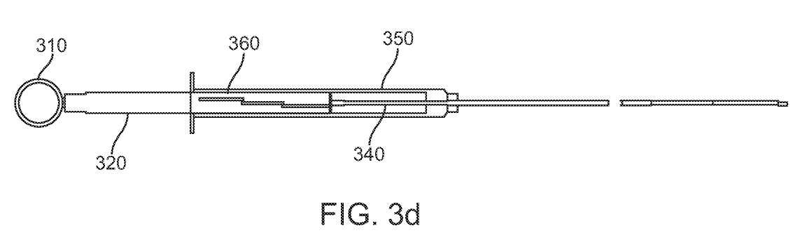

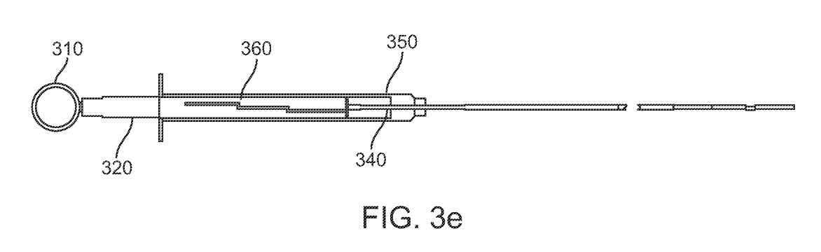

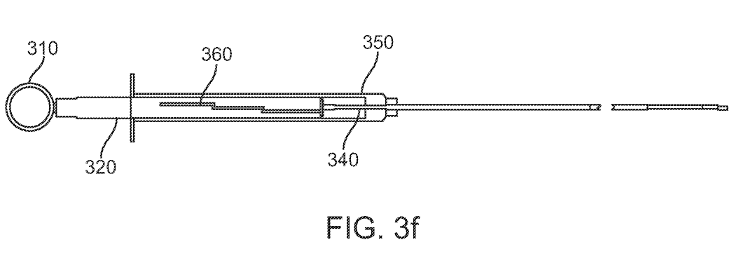

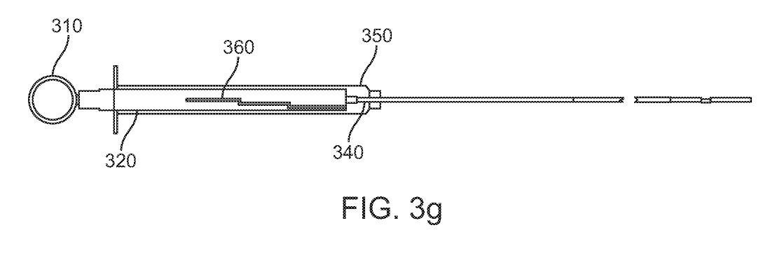

FIGS. 3a through 3g show an alternative embodiment delivery tool 300, comprising a plunger element 340 which moves forward via a rotation rod mechanism. As shown in FIG. 3a, this second embodiment comprises a rotation ring 310, a rotation rod 320, a pin 330, a plunger element 340, a housing 350 which is configured to move slidably along the rotation rod 320, a catheter 100, and one or more implants 105 stored within the distal part of the catheter 100 lumen. Optionally, the rotation rod housing 350 is also configured to stabilize the proximal opening of the catheter 100 to allow the plunger element 340 to move into the catheter 100 lumen.

The rotation rod 320 comprises at least one groove 360, as shown in FIG. 3b. In one embodiment, the groove 360 forms a zig-zag pattern comprising major sub-grooves 360a and minor sub-grooves 360b. The major sub-grooves 360a are oriented coaxially to the rotation rod 320. The minor sub-grooves 360b connect the major sub-grooves 360a and are oriented non-coaxially to the rotation rod 320. Additionally, the housing 350 comprises at least one pin 330 (as shown in FIG. 3a) configured to move within the groove 360 as the rotation rod 320 moves slidably within the housing 350. Preferably, the lengths of the major sub-grooves 360a correspond to the length of one implant 105, such that movement of the rotation rod 320 along a major sub-groove 360a will be sufficient to deploy one implant 105.

To deploy the first implant, a user pushes the rotation rod 320 in a distal direction such that the pin 330 slides along the first (i.e. the most proximal) major sub-groove to the first minor sub-groove, as shown in FIG. 3c. As the rotation rod 320 moves slidably into the housing 350, the plunger element 340 moves forward in a distal direction and into the catheter 100 lumen, thereby causing the plunger element 340 to expel and deploy an implant at the distal end of the catheter 100.

Thereafter, as shown in FIG. 3d, either the housing 350 or the rotation rod 320 is rotated such that the pin 330 slides along the minor sub-groove to the next major sub-groove, positioning the delivery tool 300 for the next implant delivery.

Thereafter, as shown in FIG. 3e, the rotation rod 320 is pushed once such that the pin 330 slides in a distal direction along the second major sub-groove to the second minor sub-groove. As the rotation rod 320 moves into the housing 350, the plunger element 340 moves forward into the catheter 100 lumen, causing the plunger element 340 to deploy the next implant. A third implant may be deployed by repeating this process, as shown in FIG. 3f. This process may be repeated as many times as desired to deploy subsequent implants, and is only limited by the number of implants loaded in the catheter 100 and the number of major and minor sub-grooves 360a and 360b.

While the above is a complete description of the preferred embodiments of the invention, various alternatives, modifications, and equivalents may be used. Therefore, the above description should not be taken as limiting the scope of the invention which is defined by the appended claims.

What is claimed is:

1. A system for delivering multiple implants into body passageways, comprising:
a catheter comprising a proximal and distal end, the distal end configured to receive and store a plurality of implants; and
an actuatable delivery tool coupled to the proximal end of the catheter;
wherein the system is configured to expel one implant out of the distal end of the catheter with each actuation of the delivery tool.

2. The system of claim 1, further comprising:
a plunger element coupled to the delivery tool and extending through the catheter, thereby allowing the delivery tool to mechanically communicate with the plurality of implants.

3. The system of claim 2, wherein the delivery tool comprises a spring-loaded slidable element for actuating the delivery tool.

4. The system of claim 3, wherein compression of the spring-loaded slidable element causes the slidable element to subsequently return to its initial position as it incrementally moves the plunger element in a distal direction, thereby expelling an implant.

5. The system of claim 4, wherein the plunger element comprises a plurality of indentations, and wherein the slidable element comprises a multiplicity of leaf springs for engaging with the indentations and incrementally moving the plunger in the distal direction.

6. The system of claim 5, wherein each incremental movement of the plunger element expels one implant.

7. The system of claim 2, wherein the delivery tool comprises a rotation rod for actuating the delivery tool.

8. The system of claim 7, wherein moving the rotation rod in a distal direction causes an incremental movement of the plunger element in the distal direction, thereby expelling an implant.

9. The system of claim 8, wherein the delivery tool further comprises a housing, and wherein the rotation rod is configured to move slidably within the housing.

10. The system of claim 9, wherein the housing comprises a non-linear groove, and the rotation rod comprises a pin configured to fit within and move slidably along the groove.

11. The system of claim 10, wherein the groove comprises major sub-grooves arranged coaxially with the rotation rod and minor sub-grooves arranged non-coaxially with the rotation rod, with the lengths of the major sub-grooves corresponding to the lengths of the implants and the minor sub-grooves serving as stops between successive implant deliveries.

12. A method for delivering multiple implants into body passageways, comprising:
advancing a catheter through a body passageway of a patient to a first location, the catheter comprising a proximal and distal end, the distal end storing a plurality of implants;

13. actuating a delivery tool coupled to the proximal end of the catheter to expel a first implant at the first location;
repositioning the distal end of the catheter at a second location within the body passageway; and
actuating the delivery tool to expel a second implant at the second location.

14. The method of claim 12, wherein the actuating steps comprise compressing a slidable element of the delivery tool.

15. The method of claim 13, wherein the compressing step comprises activating a spring-loaded mechanism that causes the slidable element of the delivery tool to return to its initial position as it moves a plunger element in a distal direction to expel an implant.

16. The method of claim 14, wherein the plunger element comprises a plurality of indentations, and wherein the slidable element comprises a plurality of leaf springs for engaging with the indentations and incrementally moving the plunger in the distal direction.

17. The method of claim 15, wherein the actuating steps comprise moving a rotation rod in a distal direction.

18. The method of claim 16, wherein moving the rotation rod in the distal direction causes an incremental movement of a plunger element in the distal direction, thereby expelling an implant.

19. The method of claim 17, wherein the rotation rod comprises a pin and is configured to move slidably within a housing, and wherein moving the rotation rod in the distal direction causes the pin to move slidably along the groove.

20. The method of claim 18, wherein the groove is non-linear and comprises major sub-grooves arranged coaxially with the rotation rod and minor sub-grooves arranged non-coaxially with the rotation rod, with the lengths of the major sub-grooves corresponding to the lengths of the implants and the minor sub-grooves serving as stops between successive implant deliveries.
